(19) United States
(12) Patent Application Publication
(10) Pub. No.: US 2015/0330938 A1
(43) Pub. Date: Nov. 19, 2015

(54) METHOD AND APPARATUS FOR MONITORING GAS CONCENTRATION

(71) Applicant: ENVIRONMENTAL MONITORING AND CONTROL LIMITED, Stafford, Staffordshire (GB)

(72) Inventors: Mark Anthony Steele HENSON, Stafford, Staffordshire (GB); Matthew Paul HILLS, Stafford, Staffordshire (GB)

(73) Assignee: Environmental Monitoring and Control Limited, Stafford,Staffordshire (GB)

(21) Appl. No.: 14/649,727

(22) PCT Filed: Dec. 5, 2013
(86) PCT No.: PCT/GB2013/000529

§ 371 Date: Jun. 4, 2015

(30) Foreign Application Priority Data
Dec. 7, 2012 (GB) 1222081.0

(51) Int. Cl. G01N 27/407; G01N 33/00; G01N 27/409; G01N 27/404

(52) U.S. Cl. CPC G01N 27/407; G01N 27/4045; G01N 33/005; G01N 27/409

(57) ABSTRACT

A probe incorporates a sensor for measuring a concentration of a gas in solution in a fluid medium. The sensor (12) is housed in or extends into a measurement chamber (62) of the probe. The measurement chamber is separated, in use, from the fluid medium by a porous wall portion (64) through which the gas, but not the fluid medium, can diffuse. A gas feed is connected to the measurement chamber for forcing, in use, a purge gas or a calibration gas through the porous wall portion, outwardly from the measurement chamber. An electrical heater (40) is provided for heating the sensor to an elevated temperature during storage. The sensor comprises a solid electrolyte carrying a measurement electrode and a reference electrode on opposite faces thereof, and connections are provided for applying a voltage across the solid electrolyte during storage of the probe.

The invention relates to a method and an apparatus for measuring the concentration of a gas, in particular the concentration of a gas in a fluid medium, such as a liquid or gaseous medium. For example, the invention may find application in monitoring the concentration of a gas in solution in a fluid medium.

Known probes and sensors for measuring the concentration of gas dissolved in fluid media are described in documents such as published patent applications WO2004/025289, WO2006/037992, WO2007/042805, and WO2010/067073, all of which are incorporated herein by reference, in their entirety. These particular documents describe probes comprising electrolytic sensors, primarily for the determination of hydrogen concentration in fluid media at elevated temperatures. Each sensor comprises a proton-conducting solid electrolyte and a metal/hydrogen reference standard contained within a sealed reference chamber on one side of the solid electrolyte. A reference electrode and a measurement electrode are positioned, or coated, on opposite sides or faces of the solid electrolyte. The side of the electrolyte carrying the reference electrode is exposed to the reference standard and the other side, outside the reference chamber and carrying the measurement electrode, is exposed to a hydrogen concentration to be measured.

Similar probes may be constructed for the measurement of other gases, such as oxygen. In that case, an oxygen reference standard and an oxygen-ion-conducting solid electrolyte would be used, as would be understood by the skilled person.

All of the hydrogen sensors described above use a solid-state, metal-hydrogen reference material contained within a sealed chamber on one side of the solid electrolyte. An alternative type of electrolytic sensor uses a gaseous reference, usually provided by supplying gaseous hydrogen (or a gas comprising a known concentration of hydrogen in an inert carrier gas) to the reference chamber. Examples of such probes are described in patent publication EP0544281 of Tokyo Yogyo KK, which is incorporated herein by reference, in its entirety.

An electrolytic sensor may conveniently be mounted at an end of a suitable support, as described in WO2006/037992 or WO 2010/067073, to form a probe. The end of the probe carrying the sensor may then be immersed in or contacted with a fluid medium, for example, at high temperature, in order to measure a gas concentration within the fluid medium.

In all of the probes described above, the sensor is arranged so that the surface of the solid electrolyte carrying the measurement electrode is exposed to the concentration of the gas to be measured. In many applications, such as the measurement of the concentration of a gas in solution in a molten metal, it may be necessary to avoid contact between the fluid medium (the molten metal) and the solid electrolyte, as the fluid medium may chemically attack the solid electrolyte.

Probe designs for such applications may therefore incorporate a measurement chamber separated from the fluid medium by a porous or permeable material through which the gas but not the fluid medium can pass. When the end of the probe is immersed in the fluid medium, the gas can therefore diffuse into the measurement chamber until it reaches an equilibrium concentration related to its concentration in the fluid medium. The surface of the solid electrolyte carrying the measurement electrode is exposed to the measurement chamber so that the concentration of the gas in the measurement chamber can then be measured.

Although some prior-art probes of these types show promising performance, it is desirable to improve probe reliability and lifetime, and to improve probe response time. The invention aims to address these problems.

### SUMMARY OF INVENTION

The invention provides a probe, a probe sleeve, and methods for assembling and operating a probe as defined in the appended independent claims, to which reference should now be made. Preferred or advantageous features of the invention are set out in dependent sub-claims.

In a first aspect, the invention may therefore provide a probe for sensing or measuring a concentration of a gas in solution in a fluid medium, in which a measurement chamber is defined within a wall of the probe, the wall comprising a porous wall portion for, in use, contacting the fluid medium such that the gas but not the fluid medium can pass, or diffuse through, the porous wall portion into the measurement chamber. The probe comprises a sensor for sensing or measuring a concentration of the gas (the measurand gas) in the measurement chamber. The probe further comprises a purge-gas feed couplable, in use, to a source of a purge gas for forcing the purge gas into and through the measurement chamber and through the porous wall portion, outwardly from the measurement chamber.

Advantageously, the porous wall portion of the probe may be of porous graphite or any other suitable material which is sufficiently inert in contact with the fluid medium and through which the gas can diffuse. The fluid medium may be a molten metal, such as aluminium, copper or zinc, or an alloy comprising aluminium, copper or zinc. Alternatively, the fluid medium may be a molten glass or the like. The gas in solution in the liquid may, for example, be hydrogen or oxygen. In a particularly-preferred embodiment, the liquid may be aluminium (such as pure aluminium or an aluminium alloy) and the gas may be hydrogen.

During use of the probe, an end of the probe may thus be brought into contact with the fluid medium. Before a measurement of gas concentration is made, the purge-gas feed is openable to supply a volume of the purge gas to the measurement chamber. The purge-gas feed is then closable, to seal the measurement chamber except at the porous wall portion. The measurand gas may then diffuse through the porous wall portion into the measurement chamber. During a preferred sampling time, the concentration of the measurand gas may increase until it reaches an equilibrium concentration in the remaining purge gas in the measurement chamber. The equilibrium concentration depends on the concentration of the gas in solution in the fluid medium. The sensor can measure the concentration of the gas in the measurement chamber.

The inventors have found that before a measurement is made it is particularly advantageous to supply through the purge-gas feed a volume of purge gas which is significantly larger than volume of the measurement chamber, so that a very large proportion of the supplied purge gas is forced through the porous wall portion, outwardly from the measurement chamber. In addition, the volume of the measurement chamber should be as small as possible. The smaller the volume of the measurement chamber, the smaller is the volume of measurand gas which has to diffuse through the porous wall portion in order to reach an equilibrium concentration in the measurement chamber, and the shorter the time required for this diffusion. This improves the response time of the probe.

Advantageously, therefore, the volume of the measurement chamber may be less than 1 ml, and preferably less than 0.5 ml, 0.25 ml or 0.15 ml.

For such a measurement chamber, the inventors have found that before a measurement is made, the purge-gas feed may advantageously be openable to supply a volume of purge gas of at least 50 ml, and preferably 100 ml or 200 ml or 300 ml or 500 ml. These volumes are as measured at atmospheric pressure, although the purge gas must be supplied to the purge-gas feed at elevated pressure, for example between 0.8 and 2.5 bar, or between 1.0 and 2.0 bar, in order to force it into the measurement chamber and through the porous wall portion.

The inventors have additionally found that the volume of purge gas should preferably be forced rapidly through the measurement chamber and the porous wall portion, in order to achieve the most repeatable results in subsequent measurement of the measurand gas concentration. Advantageously, therefore, the volume of purge gas described above should be supplied within a duration of 20 s to 60 s, or 30 s to 40 s. This may require a purge-gas flow rate of 1.7 ml·s⁻¹ or more, and preferably of as much as, or more than 3.4, 5.0, 6.7 ml·s⁻¹ or 8.3 ml·s⁻¹.

The desirable rapid purge-gas flow rate may also be quantified in terms of the rate at which the purge gas passes through the porous wall portion, per unit area of the porous wall portion. The rate of flow through the area of the porous wall portion should be at least 0.04, 0.08, 0.13, 0.16 or 0.2 ml·s⁻¹·mm⁻². All of these gas volumes are as measured at atmospheric pressure although, as described above, elevated pressures must be used during the supply of purge gas.

The volume of purge gas required to optimise the repeatability of subsequent measurement or the measurand gas may also be expressed in terms of the volume of the measurement chamber. Thus, the volume of purge gas supplied within the preferred duration of 20 s to 60 s, or 30 s to 40 s, as measured at atmospheric pressure, should be at least 50 times the volume of the measurement chamber and preferably more than 500 times or 1000 times the volume of the measurement chamber.

After the desired volume of purge gas has been supplied, the purge-gas feed is closed for measurement of the concentration of the gas in the measurement chamber. When the purge-gas feed is closed, the measurement chamber remains filled with the purge gas, as the measurand gas diffuses into the measurement chamber through the porous wall portion. Advantageously, when the purge-gas feed is closed, the closure of the feed may be positioned as close to the probe, or as close to the measurement chamber, as possible. This may advantageously minimise the effective volume of the measurement chamber into which the measurand gas will diffuse, and therefore improve (decrease) the response time of the probe during gas measurement.

The inventors have found that the supply of a large volume of purge gas significantly improves the repeatability of gas measurement, but the reason for this is not fully understood. It is believed that the flow of purge gas may remove moisture, or humidity, from the measurement chamber but it is surprising that such a large volume of purge gas, supplied at a high rate, is required to achieve the advantageous results observed by the inventors. It has been found that the purge gas should be dry, containing substantially no humidity or moisture. This would conventionally be the case for a pressurised, bottled nitrogen or an inert gas.

The purge gas is preferably nitrogen or an inert gas such as argon. In any event, however, the purge gas should be selected so that accurate measurement of the measurand gas concentration can be achieved. During measurement, the measurement chamber may advantageously contain a concentration of the measurand gas within the purge gas remaining in the measurement chamber, and the presence of the purge gas should not adversely affect the measurement.

The purge gas may also be supplied after measurand gas measurements have been made and before the probe is stored, to flush out the measurement chamber and the porous wall portion. This process may involve the same ranges of purge gas volumes, pressures and supply times as were used before gas measurements were made.

In a second aspect of the invention, a probe comprises a calibration-gas feed for supplying a calibration gas to the measurement chamber. In the same way as for the purge-gas feed described above, the calibration-gas feed may be couplable, in use, to a source of a calibration gas for forcing or supplying the calibration gas into the measurement chamber and, if required, through the porous wall portion, outwardly from the measurement chamber. A probe may comprise a calibration-gas feed and a separate purge-gas feed or the same gas feed may be selectively couplable to a source of a calibration gas or a purge gas. In any event, however, the probe is preferably controlled so that a calibration gas and a purge gas are not supplied to the measurement chamber at the same time. As for the purge gas, the calibration gas should preferably be dry, containing substantially no moisture or humidity.

Operation using a calibration gas may be as follows.

A calibration gas may consist of the measurand gas or of a predetermined, known concentration of the measurand gas in a further gas which does not affect the measurement of the measurand gas concentration. Typically, this may be nitrogen or an inert gas. When calibration of the sensor is required, the calibration gas may be supplied to the measurement chamber, in a sufficient volume to ensure that the measurement chamber is filled with the calibration gas. This may involve flushing a small excess of the calibration gas through the measurement chamber, outwardly through the porous wall portion. The calibration-gas feed is then optionally closed while the sensor measures the concentration of measurand gas in the calibration. The result of the measurement may enable re-calibration of the probe, or confirm correct operation of the probe.

The ability to carry out a calibration check using a calibration gas, preferably immediately before a real measurand gas measurement is made, may provide a particular advantage in allowing an operator to ensure that a probe is functioning correctly before measurements are made. A basic check may be made by taking a measurement from the sensor while a purge gas is supplied to the measurement chamber, at which point the measurement chamber will contain no measurand gas. The use of a calibration gas, however, allows a measurement to be made of a non-zero concentration of the measurand gas, for additional confidence that the probe is operating correctly. Advantageously a check may involve measurements of both these types.

The use of a calibration gas containing a non-zero concentration of the measurand gas may have particular value in checking a fault condition of a sensor. For example, a cracked electrolytic sensor may provide a sensor output of 0 mV, regardless of the measurand gas concentration in the measurement chamber. If the measurement chamber is filled with a purge gas containing 0% of the measurand gas, then an output of 0 mV would be expected if the sensor is fully functioning. (A low sensor reading may be expected in reality, if a small quantity of the measurand gas is able to diffuse into the measurement chamber against the outward flow of purge gas. This will depend on the purge gas flow rate and the diffusion rate of the measurand gas.) However, if the probe is operated so that the measurement chamber is filled with a calibration gas containing a known concentration of the measurand gas, and the sensor still reads 0 mV, then a faulty sensor can be diagnosed.

In some operating environments, probes comprising electrolytic sensors are exposed to aggressive conditions. For example, a probe embodying the present invention, for example for measuring hydrogen concentration in molten aluminium, may be expected to survive more than one hundred, and preferably several hundred, dips into high-temperature molten metal, and immersion for many hours in the molten metal. An electrolytic sensor contains various ceramic components and there is a risk that it may crack, ending its life. It is then very important that an operator can rapidly detect failure of such a sensor at the end of its life. The calibration and checking process described above may enable this.

In a further aspect of the invention, a calibration gas may be used instead of the purge gas, as part of the preparation of a probe for making a measurement. As described above, to purge the probe effectively before measurement, a sufficient volume of the purge gas may be forced through the measurement chamber and outwardly through the porous wall portion. As described above, this advantageously involves passing through the measurement chamber a volume of purge gas which is many times greater than the volume of the measurement chamber, within an advantageously short time, of 60 s or less. Instead of the purge gas, a calibration gas may be used in the same way to purge the measurement chamber, for example to remove humidity from the measurement chamber and the sensor, and to prepare the porous wall portion for the inward diffusion of measurand gas. If a calibration gas is used to purge the measurement chamber, a calibration measurement may simultaneously be made using the sensor. Where a calibration gas is used as a purge gas in this way, the calibration gas may be considered to be an embodiment of a purge gas as described and claimed in this document.

If the calibration gas is used for purging the measurement chamber, it may subsequently be necessary to pass a smaller volume of purge gas through the measurement chamber, in order to reduce the concentration of the measurand gas in the measurement chamber to zero before real measurements of the gas concentration in the fluid medium can be made. This would, however, require a smaller volume of the purge gas than was required to carry out the initial purging process.

Alternatively, if the calibration gas contains a smaller concentration of the measurand gas than is expected to be present in the fluid medium, the calibration gas can remain in the measurement chamber while the greater concentration of gas passes into the chamber from the fluid medium.

A suitable calibration gas for a hydrogen sensor might, for example, comprise 1% hydrogen or 0.5% hydrogen in nitrogen or in an inert gas.

In an alternative embodiment, more than one calibration gas containing different concentrations of the measurand gas may be used sequentially to calibrate or check the sensor output, preferably across a full span of measurement conditions. Such measurements could be combined, if the probe comprises a heater as described below, with measurements at different temperatures in order to calibrate or verify the sensor output across measurement conditions varying in both temperature and measurand gas concentration.

In a preferred measurement protocol, a probe embodying these aspects of the invention may be controlled as follows. The purge gas may be supplied to the probe before, during and/or after the probe is brought into contact with the fluid medium. Preferably, the purge gas is supplied at least for a period of time after the probe is brought into contact with the fluid medium. The calibration gas may then be supplied in a sufficient volume, to fill the measurement chamber, and a calibration measurement taken using the sensor. A further volume of purge gas may then be supplied to flush the calibration gas out of the measurement chamber. The purge-gas feed should then be closed to allow the measurand gas to diffuse through the porous wall portion into the measurement chamber for measurement by the sensor as described above.

In relation to a further aspect of the invention, the inventors have found that a problem arises with the storage of probes comprising electrolytic gas sensors. After storage, it is found that the performance of such probes subsequently used for gas measurements may be seriously degraded. It may be important for an appropriate very low level of humidity to be maintained in the region of the solid electrolyte, particularly if the probe is used for measuring hydrogen concentration; if the humidity is too low, then the electrolyte conductivity may be adversely affected. However, if a probe containing excessive humidity or moisture is immersed in, for example, molten aluminium containing dissolved hydrogen, the presence of the humidity in the probe may adversely affect the measurement of hydrogen concentration or even cause damage to the probe.

A third aspect of the invention addresses this problem using a probe as described above, having a purge-gas inlet, or feed. During storage, the purge gas (typically nitrogen or an inert gas) may be provided, preferably at a low flow rate through the purge-gas feed into the measurement chamber. The purge gas may thus surround the sensor and prevent ingress of gas or humidity from the atmosphere through the porous wall portion into the measurement chamber.

The rate of flow of the gas should be low, in order to reduce gas consumption during storage, but should be sufficient to prevent ingress of gas from the surrounding environment into the measurement chamber. For a given probe, a predetermined minimum gas flow rate may be required to achieve this, and may be determined in view of the probe size and geometry, or by experiment. For example, the minimum flow rate may correspond to a small pressure elevation, of 0.1 bar or 0.05 bar, in the measurement chamber as compared to the ambient atmospheric pressure. A suitable flow rate may be between 1 and 100 ml·min⁻¹, or between 2 and 50 ml·min⁻¹ (as measured at atmospheric pressure). A small oversupply of gas through the purge-gas feed, above the minimum flow rate, may then be maintained to ensure that gas ingress is avoided. Thus, the gas flow rate or pressure may be 10% or 25% or 50% higher than the minimum required rate or pressure.

In a fourth aspect of the invention, the probe may be provided with a heater capable of raising the temperature of the electrolytic sensor, preferably by more than 50 C or 100 C or 150 C or 200 C, above ambient temperature. Temperature rises in the range of 50 C to 180 C or 200 C, or in the range 80 C to 120 C or 150 C, could be used. The temperature rise will typically be above room temperature, but the heater may also be usable during immersion of the probe in the fluid medium, in which case the heater may increase the sensor temperature above that of the fluid medium.

The heater may advantageously be an electrical heater, couplable to an electrical power supply.

In a preferred embodiment, the probe may comprise a thermocouple, with the thermocouple junction in the vicinity of the electrolytic sensor. The thermocouple may be usable as described in WO2010/067073 (which is incorporated herein by reference in its entirety) to monitor the temperature of the electrolytic sensor during gas-concentration measurement. Such temperature readings may be used to remove or reduce any variations in gas concentration measurements caused by temperature variations, for example with reference to a look-up table.

In this preferred embodiment, the thermocouple in such a probe may additionally be used as the heater, by applying a sufficient voltage to the thermocouple to raise its temperature.

If probe heating is required during gas measurement, it may be important not to impair the ability of the thermocouple to measure the temperature of the electrolytic sensor. This may be achieved by temporarily switching off the heating power supply to the thermocouple, for a time short enough to avoid significant temperature variation while the power supply is switched off, while temperature measurements are made.

Advantageously, the heater may be used for several different purposes.

During storage of the sensor, typically in normal (uncontrolled) atmospheric conditions at room temperature, the heater may be activated to raise the temperature of the probe and, in particular, of the electrolytic sensor during storage. Advantageously, this may prevent the build up of humidity or moisture on or within the probe. To achieve this, the probe temperature may be raised to a temperature in the range of 50 C to 180 C, or 200 C, or between 80 C and 120 C or 150 C. The probe may be thermally insulated during storage in order to reduce the power consumption of the heater.

The inventors have tested probe storage at up to 200 C, achieved by applying 12 V to a thermocouple in a probe.

The inventors have found that heating the probe during storage is extremely effective in preventing degradation of the probe. In tests using a probe of the type illustrated in FIGS. 1 to 4 below, probes were stored either heated or unheated in normal ambient (uncontrolled) conditions. After 12 hours storage, the unheated probe took 10 minutes to provide gas measurements (of hydrogen concentration in aluminium) while the heated probe took only one minute.

Heating the probe during storage may be used in combination with the provision of a purge gas at a low flow rate through the measurement chamber as described in the third aspect of the invention described above.

In an alternative embodiment, a probe may be stored unheated, optionally with a protective flow of purge gas, and the heater used after storage to preheat the probe before immersion in the fluid medium to make a measurement.

Preheating may be carried out for example for a period of 1 to 20 minutes, or 2 to 10 minutes, before immersion. Heating the probe in this way, whether or not the probe was previously heated continuously during storage, may advantageously drive any excess humidity or moisture out of the measurement chamber before gas measurements are made.

A third application of the heater may be used in calibrating or checking the probe. The heater may be used to vary the temperature of the sensor so that measurements of the concentration of the measurand gas in one or more calibration gases, for example containing different gas concentrations, may be made at more than one predetermined temperature of the sensor. This information may be used to calibrate or re-calibrate the sensor or to confirm correct operation of the sensor before real measurements of measurand gas concentration are made.

In a fifth aspect of the invention, the storage condition of the probe may advantageously be improved by applying an electrical voltage between the measurement electrode and the reference electrode of the sensor during probe storage. The voltage is preferably applied with a polarity opposite to the voltage generated by the sensor during gas measurement. The mechanism by which this process works is not fully understood but the inventors' observations indicate that storing a probe with a voltage applied across the solid electrolyte in this way advantageously conditions the probe for future use, so that on subsequent immersion into a fluid medium, the response time of the probe for gas measurement is significantly improved.

This aspect of the invention may be combined with the provision of an electrical heater in the probe, as described above. Thus, for example, an electrical-supply voltage coupled to an electrical heater may, at the same time, be applied across the measurement electrode and the reference electrode of the sensor.

Preferably, a single electrical lead, or connection, may then be used to connect the electrical power supply to one of the electrodes of the sensor and also to the heater. This use of a common electrical connection to the heater and the sensor may advantageously reduce the number of electrical connections or leads required within the probe.

The physical structure of the probe may be any structure which enables the functionality described above, for implementing any of the individual aspects of the invention or any combination of multiple aspects of the invention. Each of the aspects may be implemented either individually or in combination with one or more other aspects, to provide synergistic advantages.

In a preferred embodiment, a probe may comprise an electrolytic sensor mounted at a first end of a probe, which may be termed the measurement end. The probe may extend from the measurement end to a support end, which may be secured to a probe-manipulating apparatus, such as an automated apparatus, or may comprise a handle, for manual operation. The probe may be handled from the support end and the measurement end immersed in the fluid medium. This structure may be important if the fluid medium is at high temperature or is chemically aggressive.

An outer surface of the probe, or at least the portion of the probe which will be exposed to the fluid medium, comprises a probe sleeve or sheath. The sleeve is preferably of a material which is inert in the presence of the fluid medium.

An end of the probe sleeve, at the measurement end of the probe, may comprise a porous wall portion which, when in contact with the fluid medium, allows the measurand gas but not the fluid medium to diffuse through the porous wall portion into the measurement chamber. The measurement chamber is preferably defined within an end portion of the probe sleeve, and the sensor preferably forms a boundary or wall of the measurement chamber, or extends into the measurement chamber, to enable measurement of the gas concentration in the measurement chamber.

An end of the probe sleeve may conveniently be in the form of a removable cap, which may incorporate the porous wall portion and may advantageously define a wall of the measurement chamber.

The electrolytic sensor requires two electrical leads, or contacts, one connected to each of the reference electrode and the measurement electrode. These may be implemented in any convenient manner, so that the sensor voltage can be detected during gas measurement. For example, both the reference electrode and the measurement electrode may be connected to electrical conductors, or leads, extending to the support end of the probe, where a connection block may be provided for making electrical connections to suitable electronic measurement equipment. Alternatively, if the fluid medium is an electrical conductor (such as a molten metal) then an electrical connection to one of the sensor electrodes, usually the measurement electrode, may be made through the fluid medium.

At a portion of the probe sleeve which is preferably spaced from the fluid medium during use, and is optionally at the support end of the probe, a purge-gas feed and/or a calibration-gas feed may be provided, for coupling one or more gas supplies to an internal volume of the probe sleeve. The internal volume of the probe sleeve may be connected to the measurement chamber so that gas supplied to the gas feed or feeds enters or flows to the measurement chamber. The probe may comprise a valve or tap for opening and closing the or each gas feed, preferably positioned close to the probe sleeve or the measurement chamber so that when the valve(s) or tap(s) are closed, the effective volume of the measurement chamber is minimised.

Gas supplies may conveniently be from pressurised containers, or bottles, for supplying suitably pure, dry gases.

The probe may comprise a heater, preferably in the region of the sensor. The heater is preferably an electrical heater, couplable to a power supply. Conveniently, electrical leads for supplying power to the heater may extend within the probe towards the support end of the probe. Conveniently, a contact block or other contact arrangement may be provided at the support end of the probe for coupling the probe to a suitable electrical power supply. This may enable electrical power to be supplied to the heater and/or an electrical voltage to be applied across the solid electrolyte of the sensor as described above. The contact block may also provide electrical connections to the measurement electrode and the reference electrode to allow the sensor output to be detected during gas measurements.

A control system or controller may be provided to enable implementation of the various aspects of the invention described above. For example, a controller may control the application of electrical power to a heater, if present, and across the electrodes of the sensor during storage, if desired. The controller may also monitor the voltage output of the sensor during measurement and, if desired, during checking and/or calibration. The control system or controller may also control the supply of purge gas and/or calibration gas or gases to the probe, optionally at the same time as or in conjunction with controlling electrical inputs and outputs of the probe. Thus, for example, a controller may implement a storage mode of the probe, in which a slow flow of purge gas is supplied to the probe and/or in which the probe is heated. The controller may then implement a protocol for preparing the probe for making a measurement. This may involve a predetermined heating step, and/or a predetermined supply of a purge gas and/or a calibration gas to the probe as described above. Optionally, at the same time the controller may monitor sensor output voltages, for example to check the integrity of the probe and the sensor and/or to calibrate or re-calibrate the sensor. If, for example, a calibration gas is used, then sensor readings may be taken at appropriate times when the measurement chamber is filled with a predetermined calibration gas. If gas measurements or calibration gas measurements are to be made at different temperatures controlled using a probe heater, the controller may advantageously control and synchronise the power supply to the heater. If the probe comprises a thermocouple, the controller may monitor the temperature using the thermocouple. If the thermocouple is also used as a heater, the controller may control suitable interruptions of the heating power supply to the thermocouple to allow temperature measurements to be made.

In a preferred probe apparatus, it may be commercially important to be able to re-use or recycle components of the probe. It is anticipated, for example, that the lifetime of an electrolytic sensor may be less than the lifetime of other probe components, such as the probe sleeve and the controller. The probe may advantageously be constructed so that the sensor, or the sensor and a sensor support, are replaceable and other components are reusable.

As noted above, an end portion of the probe sleeve may be formed by a cap, which is optionally removable. The cap may, for example be of graphite and threadedly connectable to the probe sleeve. The end cap advantageously comprises the porous wall portion and in certain applications it is possible that the porous wall portion may have a limited lifetime, advantageously after many gas measurements have been taken. In that case, the probe cap may be replaceable.

An exemplary operating protocol embodying various aspects of the invention may be as follows:

1. Position the probe over the melt (for example the fluid medium may be molten aluminium)
2. Turn on purge gas (N2)

### 3. Wait 1 min

4. Dip probe into aluminium

### 5. Wait 1 min

6. Stop purge gas flow
7. Observe the measurand gas (e.g. H₂) quickly equilibrate/diffuse into the measurement chamber.
8. Measurements can continue to be made, with the probe in the fluid medium, for as long as monitoring of the gas concentration is required.
9. After measurement has finished turn on purge gas to flush probe and porous wall portion

### 10. Wait 1 min

11. Remove the probe
12. Wait until cooled down (e.g. 5 mins)
13. Then for probe storage:
a. Switch to purge gas at low flow rate;
b. Switch on heater current and turn off purge gas; or
c. Leave purge gas on preferably at reduced (low) flow rate and switch heater on for a semi-permanent storage situation.

### SPECIFIC EMBODIMENTS AND BEST MODE OF THE INVENTION

Specific embodiments of the invention will now be described by way of example, with reference to the accompanying drawings, in which:

FIG. 1 is a side view of a probe according to a first embodiment of the invention;

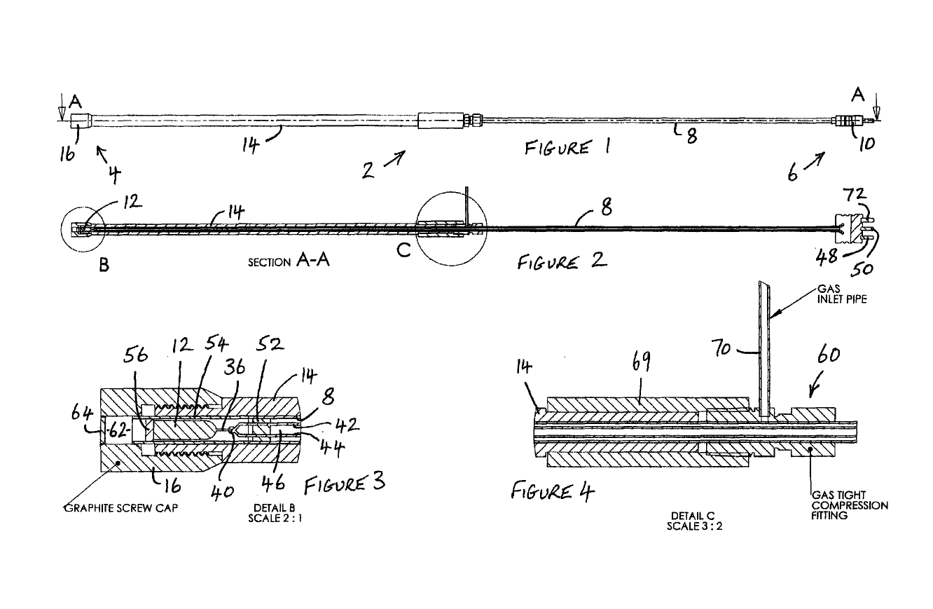

FIG. 2 is a longitudinal section of the probe of FIG. 1, on A-A;

FIG. 3 is an enlarged view of the measurement end of the sectioned probe of FIG. 2, shown at B in FIG. 2;

FIG. 4 is an enlarged portion of the ringed area C in FIG. 2;

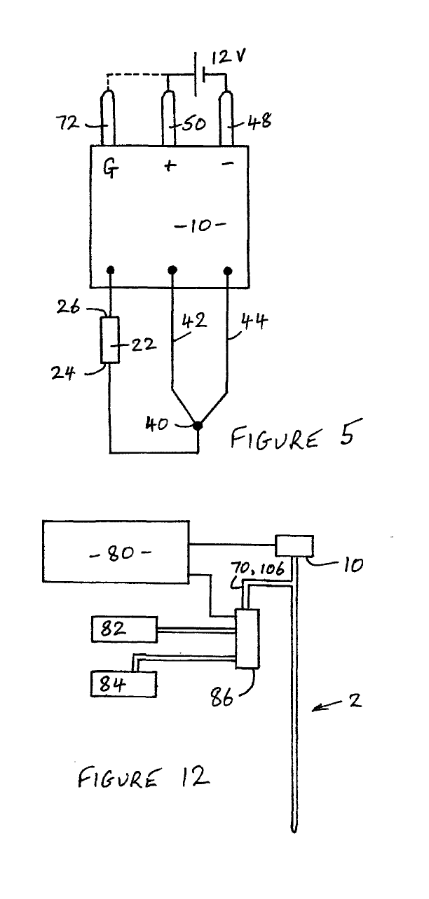

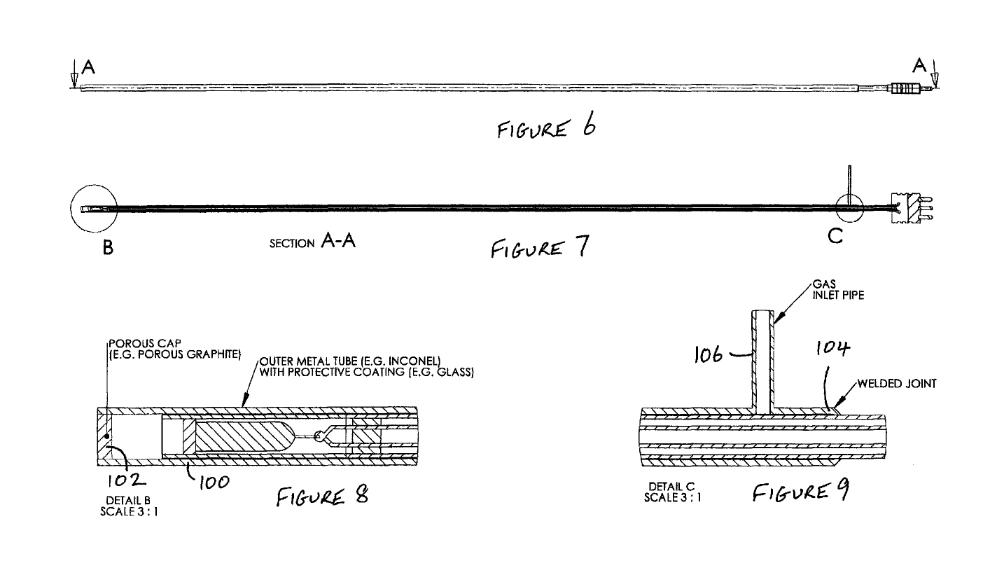

FIG. 5 is a circuit diagram showing electrical connections to the probe of FIGS. 1 to 4;

FIG. 6 is a side view of a probe according to a second embodiment of the invention;

FIG. 7 is a longitudinal section of the probe of FIG. 6, on A-A;

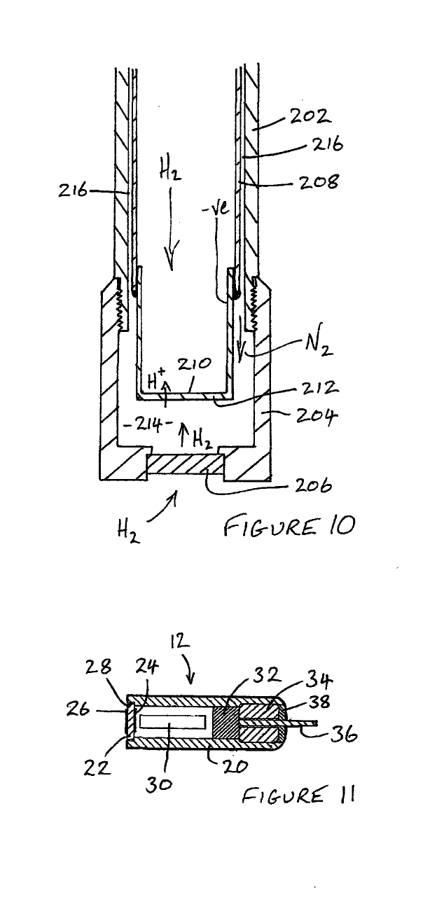

FIG. 8 is an enlarged view of the ringed area C in FIG. 7;

FIG. 9 is an enlarged view of the measurement end of the probe of FIG. 7, as shown at B in FIG. 7;

FIG. 10 is a longitudinal section of the measurement end of a probe according to a third embodiment of the invention, incorporating a gaseous hydrogen reference;

FIG. 11 is a longitudinal section of an electrolytic sensor having a solid reference material, as used in the probes of the first and second embodiments shown in FIGS. 1 to 9;

FIG. 12 illustrates a control system for a probe embodying the invention;

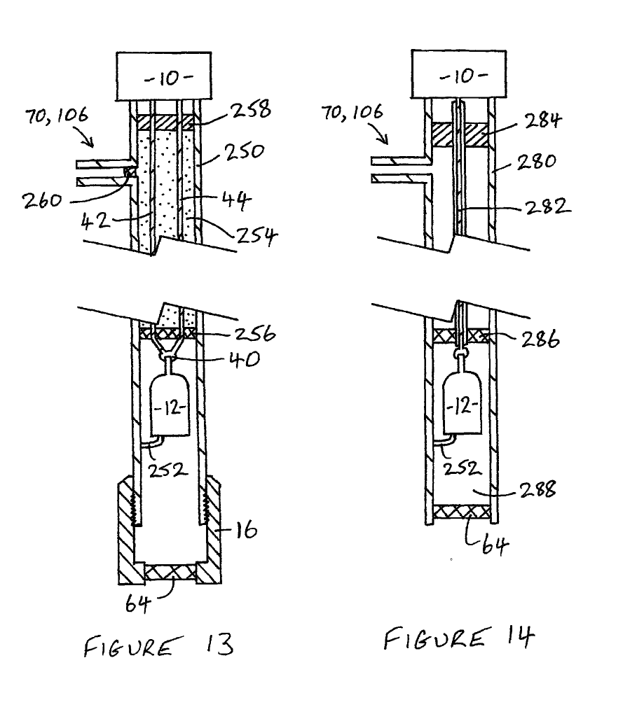

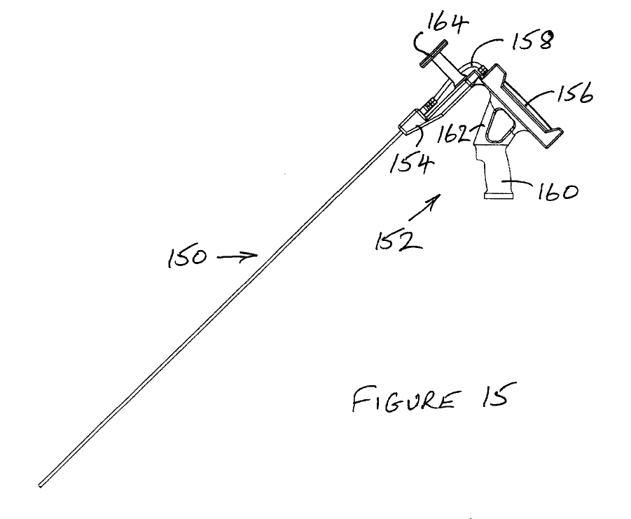

FIG. 13 is a longitudinal section of a probe according to a fourth embodiment of the invention;

FIG. 14 is a longitudinal section of a probe according to a fifth embodiment of the invention;

FIG. 15 shows a probe fitted with a handle, for manual operation, according to a sixth embodiment of the invention;

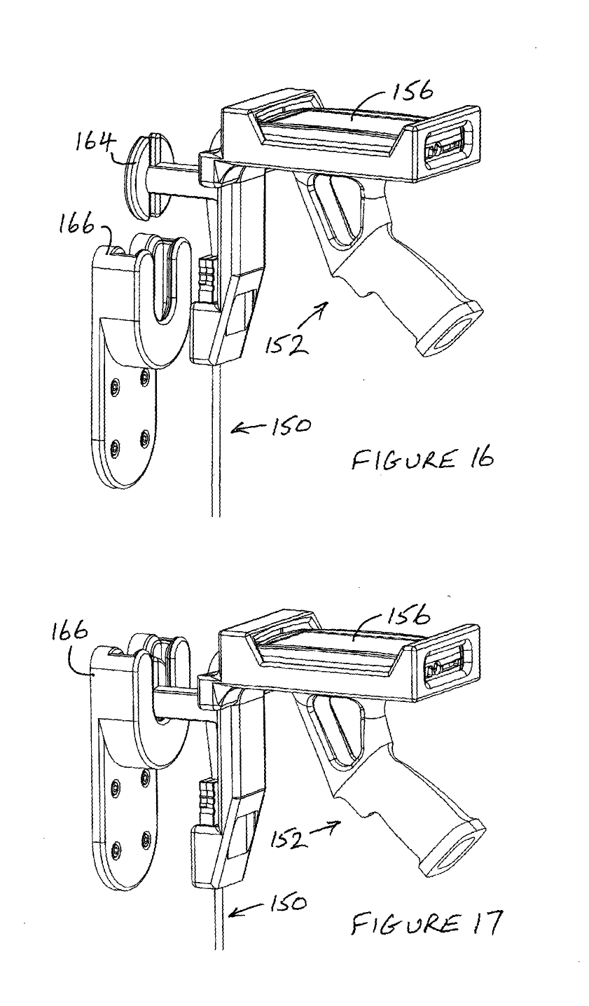

FIG. 16 is a close-up view of the handle of the probe of FIG. 15, before insertion into a storage holder; and

FIG. 17 shows the handle of the probe of FIG. 16 docked in its holder for storage.

FIGS. 1 to 5 illustrate a probe according to a first embodiment of the invention. The probe is for measuring the concentration of hydrogen in solution in molten aluminium. The probe 2 extends between a measurement end 4, designed for immersion into molten aluminium, and a support end 6. A sensor support 8, in the form of an inconel tube, extends from a contact block 10 at the support end, and carries a sensor 12 (see FIGS. 2 and 11) at the measurement end. Approximately half of the length of the sensor support 8, towards the measurement end, extends within a probe sleeve 14. The end of the probe sleeve, at the measurement end of the probe, comprises an end cap 16 within which a measurement chamber is defined as described below. The probe sleeve is of a ceramic material which is inert in relation to molten aluminium, such as SiAlON or SiN, and the end cap 16 is of graphite.

The sensor 12 is of conventional design, as taught by, for example, WO 2010/067073. The sensor is shown in section in FIG. 11. The sensor comprises a ceramic tube 20, of about 4 to 5 mm external diameter, closed at one end by a planar, proton-conducting, solid-electrolyte disc 22, for example of indium-doped calcium zirconate. The ceramic tube 20 is preferably made from undoped calcium zirconate so that its thermal expansion matches that of the electrolyte disc. A platinum reference electrode 24 is formed on the surface of the disc within the tube, and a platinum measurement electrode 26 is formed on the surface of the disc facing away from the tube. The disc is sealed to the tube using a silica-free glass 28. A metal-metal hydride reference material 30 is inserted into the tube behind the reference electrode and an electrical conductor (not shown) extends from the reference electrode along an internal wall of the tube. A volume within the tube behind the reference material is filled with an inert buffer material 32, such as Y₂O₃ powder. A sensor cap 34, preferably also of undoped calcium zirconate, is secured in the end of the tube using a silica-free glass. An electrode contact wire 36 extends through a hole in the sensor cap and makes contact with the electrical conductor extending from the reference electrode 24. The electrode contact wire is sealed in the hole using a glass seal 38, preferably of a silica-free glass. The solid electrolyte disc, the tube and the sensor cap thus form the walls of a sensor body enclosing a sealed cavity. The cavity contains the solid reference material, which generates a reference hydrogen partial pressure within the cavity. The electrode contact wire 36 extends outwardly from the sensor body, coaxial with the tube.

FIG. 3 shows an enlarged longitudinal section of the measurement end of the probe.

As shown in FIG. 3, the junction 40 of a thermocouple protrudes from an end of the sensor support tube 8. The two thermocouple leads 42, 44 extend within the sensor support tube, embedded in powdered mineral insulation 46, and terminate at two contacts 48, 50 of the contact block at the support end of the probe. The end of the sensor support tube adjacent to the thermocouple junction is hermetically sealed with a silica-free glass 52.

The thermocouple junction 40 is connected, for example by welding, to the reference-electrode contact wire 36 of the sensor 12. Either of the thermocouple leads can then be used to detect the voltage of the reference electrode of the sensor, as described in WO 20101067073.

During assembly of the probe, after connection of the thermocouple junction to the reference-electrode contact, a short length of inconel tube 54, of the same diameter as the sensor support tube, is secured to the end of the sensor support tube, for example by welding, to surround the sensor and to retain the sensor in position. The short length of inconel tube thus forms an end portion of the sensor support. A plug of graphite wool 56 is inserted into the end of the inconel tube 54, adjacent to the sensor. The graphite is highly permeable, so as not to obstruct access of gas to the measurement electrode, but provides an electrical connection between the reference electrode and the inconel tube 54. Alternatively, a welded connection between the measurement electrode and the inconel tube may be made. This forms an electrical contact to the measurement electrode, for gas concentration measurements.

At the support end of the probe, the contact block 10 is secured to an end of the sensor support tube 8 for mechanical support. In addition, the sensor support tube may be electrically connected to a terminal 72 of the contact block if the sensor support tube is to be used to form an electrical connection to the measurement electrode.

To assemble the probe, the sensor support, carrying the sensor, is inserted through a compression fitting 60 at an end of the probe sleeve spaced from the measurement end. The sensor support is inserted into the sleeve until the sensor is appropriately positioned at the measurement end of the sleeve (as described in more detail below) and the compression fitting is tightened against the outer surface of the sensor support tube, to form a gas-tight seal.

As shown in FIG. 3, in the assembled probe the sensor, supported by the sensor support tube, extends beyond the end of the SiAlON sleeve 14, and into a measurement chamber 62, defined within the graphite end cap 16. The end cap is threadedly connectable to the SiAlON sleeve, and abuts an end face of the SiAlON sleeve to provide a gas-tight joint. A cylindrical, internally-threaded, portion of the graphite cap is of high-density, gas-impermeable graphite. A cylindrical inner surface of this portion of the graphite cap defines a side wall of the measurement chamber. However, an end face of the graphite cap, at an end of the measurement chamber, comprises a porous wall portion 64 made of porous graphite. The grade of porous graphite is selected so that it is readily permeable to hydrogen but not molten aluminium.

It is important to minimise the volume of the measurement chamber so that, in use, a minimum quantity of hydrogen needs to diffuse through the porous wall portion in order to reach equilibrium in the measurement chamber. Consequently, it is important to position the sensor as close as possible to the porous wall portion. However, the inconel tube 8 has a higher coefficient of thermal expansion than the SiAlON sleeve 14, and so when the probe is immersed in molten aluminium, the relative expansion of the inconel sensor support will move the sensor, and the end of the sensor support, towards the porous wall portion. In order to allow accurate positioning of the sensor, when the probe is being assembled the graphite end cap 16 is removed, and a sensor-positioning end cap is threaded onto the SiAlON sleeve instead. The sensor support is then inserted into the sleeve until it abuts an end surface of the sensor-sleeve-positioning end cap, and the gas-tight compression fitting is secured. When the graphite end cap is then replaced on the SiAlON sleeve, the correct measurement-chamber dimensions are achieved.

The compression fitting 60 at the end of the probe sleeve is hermetically sealed to the probe sleeve by a ceramic collar 69 and incorporates a gas feed, or gas inlet pipe, 70. The gas feed is connected to a clearance space between the internal surface of the probe sleeve and the external surface of the sensor support, leading to the measurement chamber. The sensor support tube has an external diameter of 6.05 mm and the internal diameter of the SiAlON sleeve is preferably about 6.3 mm. In practice, the clearance between the sensor support and the sleeve should be sufficient to allow gas to flow from the gas feed to the measurement chamber, but sufficiently small that the volume of the space between the sensor support and the sleeve is advantageously small, and preferably significantly smaller than the volume of the measurement chamber. This may prevent the volume of the space between the sensor support and the sleeve from affecting the rate at which the measurand gas reaches equilibrium in the measurement chamber.

The clearance between the sensor support and the sleeve is preferably between 25 μm and 275 μm, and may advantageously be between 50 μm and 150 μm.

FIG. 5 shows electrical connections to the contact block N. The block comprises three connections. Two of these, connections 48 and 50, are connected to the thermocouple leads as described above. The third connection 72 is shown as being connected to the sensor support tube, which is electrically connected to the measurement electrode as described above. In an alternative embodiment, if electrical connection is made to the measurement electrode through the fluid medium (aluminium), then a connection to the aluminium may be made from terminal block connection 72.

The probe may be used in several modes of operation.

In storage, it may be important to avoid any build-up of humidity or moisture in the region of the sensor. To prevent this, an inert purge gas, or cover gas, may be supplied to the gas feed 70 at a slow flow rate. The purge gas flows slowly through the measurement chamber and out through the porous wall portion 64, preventing ingress of humidity or other components of the atmosphere into the measurement chamber. In the alternative, or in addition, an electrical voltage may be applied to connections of the terminal block, to supply a voltage across the thermocouple leads. The thermocouple then functions as a heating element, and may advantageously raise the temperature of the probe and, in particular, the sensor region. A temperature in the range of 50° C., 100° C., or 150° C. may be desirable to keep the probe dry during storage.

In addition, or in the alternative, an electrical voltage may be applied between one of the thermocouple leads and the measurement electrode, so that a positive voltage is applied to the measurement electrode, relative to the reference electrode. Application of this voltage across the solid electrolyte during storage has been found to improve performance of the probe during subsequent measurement, even when the probe has deliberately been kept in disadvantageously humid conditions during storage.

The contact block is shown in FIG. 5 with a 12 V supply connected across the thermocouple. This is in the mode of operation using the thermocouple as a heater. Applying 12 V to the thermocouple heats the sensor region of the probe to about 200 C. At the same time the connection made by the dotted line between two of the terminal block contacts 50, 72 makes these two contacts common and applies the 12 V supply in parallel across the solid electrolyte of the sensor. In other embodiments, other voltages may be used depending on the design of the heater and the desired temperature. If the heater voltage is different from the desired voltage across the electrolyte, different voltages could be applied to each. It is understood that voltages between 3 V and 20 V, or 6 V and 15 V, or 8 V and 13 V may be effective applied across the solid electrolyte during storage.

At the same time as voltages are applied to the heater and/or the electrolyte, a storage-mode purge-gas flow may be set up through the measurement chamber as described above.

When the probe is required for making a measurement, then a higher-pressure supply of purge gas may be applied to the gas feed, before, during and/or after immersion of the measurement end of the probe into the molten aluminium. It has been found that a rapid supply of purge gas through the measurement chamber and out through the porous wall portion advantageously prepares the probe for measurement. Preferably, a volume of purge gas (measured at atmospheric pressure) of several hundred times the volume of the measurement chamber should be forced through the measurement chamber within a period of about 20 seconds, about 30 seconds or less than a minute. It is believed that this process, particularly when carried out with the probe immersed in the molten aluminium, effectively clears, or cleans, the porous wall portion, and primes it for a repeatable inward diffusion of hydrogen (the measurand gas) after the purge gas flow is closed.

In previous probe designs, the inventors have found that coating an external surface of the porous wall portion in order to improve wetting by the molten aluminium has improved hydrogen transfer through the porous wall portion during measurement. However, the coating invariably wears off after repeated dips into molten aluminium. This not only damages the performance of these prior-art probes, decreasing the rate at which hydrogen diffuses through the porous wall portion, but more seriously, the change in the coating of the porous wall portion affects the repeatability of measurements on repeated dips. In relation to the present invention, the inventors have found that by carrying out the purge gas protocol described above, repeatable and rapid hydrogen transfer through the porous wall portion can be achieved without a coating having been applied to the porous wall portion. This means that the probe embodying the invention may achieve improved repeatability from one measurement to the next, throughout its lifetime which may be for hundreds of measurements/dips.

The gas feed 70 may also be used to supply a calibration gas containing a known concentration of the measurand gas, such as 10% or 5% or 1% or 0.5% or 0.25% hydrogen in nitrogen or argon, through the measurement chamber. This may be used instead of the purge gas to clean, or clear, the porous wall portion and to purge the internal volume of the probe, but when a calibration gas is provided, it may also advantageously be used to check and/or calibrate the sensor. A sensor measurement may be taken while a purge gas (containing no hydrogen) fills the measurement chamber, but this can only provide a sensor reading for zero hydrogen concentration. More accurate checking and/or calibration can be carried out using a calibration gas. Different calibration gases may also be provided to the gas feed, containing different concentrations of hydrogen in nitrogen or an inert gas, to provide more extensive calibration measurement. Calibration may be carried out using a range of calibration gases having measurand gas concentrations spanning an expected range of measurand gas measurements. At the same time, the heating function of the thermocouple may be used to vary the temperature of the sensor, so that calibration readings at different temperatures can be made. If the heater is used in this way while the probe is immersed in molten aluminium, measurements at different temperatures can still be made, but the attainable temperature range will be determined by the temperature of the aluminium. Temperatures can be monitored using the thermocouple.

After calibration using a calibration gas, it may be necessary to supply a purge gas to the gas feed in order to reduce the hydrogen concentration in the measurement chamber to zero, before gas measurements can be taken.

In a preferred embodiment, a control system, or controller, 80 as shown in FIG. 12 may be used to control parameters including one or more of the following; the type of gas provided to the gas feed (e.g. purge gas 82 or calibration gas 84), the opening and closing of the gas feed 70, 106 (using automated control valves 86), the provision of electrical power through the terminal block 10 to the heater/thermocouple, and the application of an electrical potential across the solid electrolyte. The controller may thus be programmed to implement a storage protocol involving predetermined gas and/or electrical power supplies, a calibration protocol involving predetermined gas and/or electrical power supplies together with sensor readings and thermocouple readings, a checking protocol involving predetermined gas supply and/heating combined with sensor measurement to check the integrity and functionality of the probe and sensor, and a measurement protocol or protocols involving purging, calibration, checking and/or sensor measurement and/or temperature measurement.

Probes embodying the invention may be applied in environments involving different degrees of mechanisation or automation. For example, a fully-automated probe may be supported and dipped into the aluminium by a machine, and all gas and electrical controls performed automatically. In a less automated environment, a probe may be hand-held and, for example, a purge gas or a calibration gas may be supplied to the probe under manual control. For example, a small compressed-gas cylinder or cylinders may be coupled to the probe for the supply of purge gas and/or calibration gas.

FIGS. 15 to 17 illustrate a probe incorporating a handle for manual operation of the probe. FIG. 15 shows the probe 150 secured to the handle 152. The terminal block of the probe clips into a moulding 154 of the handle for mechanical support. An electronic controller 156 with a display is supported on an upper surface of the handle for ease of viewing by an operator, and coupled by an electrical cable 158 to the terminal block. The handle comprises a handgrip 160 for an operator to hold. A miniature, refillable, compressed-gas bottle fits within the handgrip and is coupled by a tube to a gas inlet of the probe (not shown). The tube may conveniently be integrated with the cable 158 so that the probe can be conveniently coupled to the handle by a single push-fit connector 159. The gas cylinder contains a purge gas or, optionally, a calibration gas, and the gas may be admitted from the bottle into the probe by means of a valve operated by a trigger 162. In an alternative handle design, more than one gas cylinder may be accommodated to provide sources of both a purge gas and a calibration gas.

The controller 156 may enable any of the functions described herein, as performed for example by the controller 80 shown in FIG. 12. Thus, the controller 156 may implement operating protocols such as storage and measurement protocols. It may be set up to operate an automated valve for the admission of purge gas into the probe, or it may be programmed to display a request for the operator to operate the trigger 162 at appropriate times to admit purge gas.

As shown more clearly in FIG. 16, the handle comprises a storage hook or adaptor 164, protruding from a front end of the handle and engageable with a wall-mountable storage socket 166. FIG. 17 shows the handle docked with the storage socket. During storage, the probe is conveniently held away from contact with any surfaces, to prevent damage to the probe. The controller 156 may implement a storage mode of the probe, in which a heater heats the probe as described above and/or in which a voltage is applied across the solid electrolyte. Alternatively, docking the handle with the socket may automatically switch the probe into the storage mode. The storage socket may conveniently incorporate a supply of electrical power (not shown) to power the heater during storage. An external purge-gas supply may also be connected for long-term storage if desired.

In this embodiment, a hand-held probe may conveniently and reliably implement the various embodiments of the invention described herein, and may contain a suitable memory for logging gas measurements during use.

In one embodiment, the probe may be supported on a reticulated arm clamped to the side of a containment vessel containing molten aluminium. The probe may be positioned for measurement by the articulated arm, for example under computer control. A compressed-gas cylinder or a compressed gas line may be used to feed compressed gas (including purge gas and/or calibration gas) to the probe, and the probe may comprise a gas delivery tube for coupling to an external gas supply. A powered sensor cable may be provided to heat the probe through the thermocouple. A magnetic clamp may be provided for ease of positioning the probe. Provision may also be made for switching the purge gas to a calibrated hydrogen gas source, for example during insertion into the aluminium.

FIGS. 6 to 9 illustrate a probe according to a second embodiment of the invention. In this probe 100, the sensor block 10, the sensor support 8 and the sensor 12 are the same as in the first embodiment illustrated in FIGS. 1 to 4. In the second embodiment, however, the probe sleeve is different from the probe sleeve in the first embodiment. In the second embodiment, the probe sleeve 100 is a metal tube, preferably of inconel and coated with a protective coating such as a glass or ceramic coating, to protect the inconel from molten aluminium during measurement. At the measurement end, an inner wall of the metal sleeve defines the measurement chamber, and the end of the metal sleeve is closed by a porous wall portion in the form of a porous cap, such as a porous graphite disc 102 sealed into the end of the metal tube. The end of the metal-tube probe sleeve distant from the measurement end encircles and is welded to the sensor support, to form a gas-tight seal 104. Close to the welded seal, the probe sleeve is provided with a gas feed or gas inlet pipe 106.

Functionally, the probe of the second embodiment operates in the same way as for the first embodiment described above. However, the construction of the probe is simpler than in the first embodiment, and so the probe may be cheaper than the probe in the first embodiment. On the other hand, because the probe sleeve is welded to the sensor support, if the sensor fails then the entire probe may need to be replaced. In the first embodiment, if the sensor fails then the sensor support and the sensor can be withdrawn from the probe sleeve, and a new sensor and sensor support inserted, enabling re-use of the probe sleeve.

FIG. 10 is a transverse section of the measurement end of a probe 200 according to a third embodiment of the invention. The probe sleeve is of the same construction as the probe sleeve in the first embodiment, comprising a ceramic tube 202 and a graphite end cap 204 incorporating the porous wall portion 206. However, the electrolytic sensor for sensing hydrogen concentration in the measurement chamber uses a gaseous hydrogen reference, supplied through a tube 208 to the reference electrode 210 on a surface of the solid electrolyte 212 adjacent the measurement chamber 214, rather than using a solid hydrogen reference material.

In the probe 200, a space 216 between the probe sleeve and the hydrogen-containing tube 208 is used for the provision of purge gas and/or calibration gas, as described in relation to the earlier embodiments.

FIGS. 13 and 14 illustrate probes according to fourth and fifth embodiments of the invention. In these embodiments, the same reference numerals have been used as in earlier embodiments where components are unchanged.

In the embodiment of FIG. 13, a probe sleeve comprising an Inconel tube 250 extends from a terminal block 10. A graphite probe cap 16 comprising a porous wall portion 64 is threaded onto the measurement end of the Inconel tube. Within the Inconel tube, thermocouple wires 42, 44 extend from the terminal block to a thermocouple junction 40 at the measurement end of the probe. The thermocouple junction is welded, or otherwise connected, to the reference-electrode contact of a solid-hydrogen-reference electrolytic sensor 12. The measurement electrode of the sensor is connected by a wire 252 to the Inconel tube. This provides an electrical contact between the measurement electrode and the terminal block.

The thermocouple wires are insulated from each other and from the Inconel tube by a coarse-grained ceramic powder 254, which is retained in the Inconel tube between a porous plug 256 adjacent to the sensor, a hermetic seal 258 which closes the Inconel tube adjacent to the terminal block, and a porous plug 260 which prevents the insulation from entering the gas feed 70, 106.

The functionality of this embodiment is the same as for the first and second embodiments described above, in that purge gas or calibration gas can be admitted to the gas feed, and can flow into the measurement chamber and outwardly through the porous wall portion 64. The gas flows through the coarse-grained insulation material and through the porous seal 256 to enter the measurement chamber.

In the probe of FIG. 14, an Inconel tube 280 extends from a terminal block 10. An insulated wire 282 extends from the terminal block within the Inconel tube and is connected, at the measurement end of the probe, to the reference-electrode contact of a solid-hydrogen-reference electrolytic sensor 12. The measurement electrode of the sensor is electrically connected to the tube by a wire 252. A hermetic seal 284 closes the tube adjacent to the terminal block and a porous plug 286 is positioned adjacent to the sensor at the measurement end of the probe, to define a measurement chamber 288 containing the sensor and mechanically to support an end of the insulated wire 282 within the tube. The tube is closed at the measurement end by a porous wall portion 64.

Gas admitted to the gas feed 70, 106 can flow through the porous plug 286 into the measurement chamber to enable operation of the probe as described in the embodiments above. This form of probe, as illustrated in FIG. 14, does not incorporate a heater but a separate heating element could be added.

As the skilled person would appreciate, similar probes could be fabricated (using known techniques) to measure concentrations of hydrogen in other fluid media or concentrations of other gases in fluid media. In each case, however, the volume of the measurement chamber is preferably as small as possible, in order to accelerate measurement times, and the gas in the measurement chamber, adjacent to the sensor, should be sealed within the probe during measurement, in order to allow rapid equilibration with the gas in the fluid medium.

1. A probe for measuring a concentration of a gas in solution in a fluid medium, comprising;
a measurement chamber defined within the probe;
a porous wall portion for, in use, contacting the fluid medium such that the gas but not the fluid medium can pass through the porous wall portion into the measurement chamber;
a sensor in the measurement chamber or extending into the measurement chamber for measuring a concentration of the gas in the measurement chamber; and
a purge-gas feed connected to the measurement chamber and couplable, in use, to a source of a purge gas for forcing the purge gas through the porous wall portion, outwardly from the measurement chamber.

2. A probe for measuring a concentration of a gas in solution in a fluid medium, comprising;
a measurement chamber defined within the probe;
a porous wall portion for, in use, contacting the fluid medium such that the gas but not the fluid medium can pass through the porous wall portion into the measurement chamber;
a sensor in the measurement chamber or extending into the measurement chamber for measuring a concentration of the gas in the measurement chamber; and
a calibration-gas feed connected to the measurement chamber and couplable, in use, to a source of a calibration gas for supplying the calibration gas to the measurement chamber and, optionally, through the porous wall portion, outwardly from the measurement chamber.

3. A probe for sensing a concentration of a gas in solution in a fluid medium, comprising;
a measurement chamber defined within the probe;
a porous wall portion for, in use, contacting the fluid medium such that the gas but not the fluid medium can pass through the porous wall portion into the measurement chamber;
an electrolytic sensor for measuring a concentration of the gas in the measurement chamber; and
a heater for heating the electrolytic sensor.

4. A probe for sensing a concentration of a gas in solution in a fluid medium, comprising;
a measurement chamber defined within the probe;
a porous wall portion for, in use, contacting the fluid medium such that the gas but not the fluid medium can pass through the porous wall portion into the measurement chamber; and
an electrolytic sensor for measuring a concentration of the gas in the measurement chamber;
in which the electrolytic sensor comprises a measurement electrode and a reference electrode on opposite surfaces of a solid electrolyte, and the probe comprises an electrical connection for coupling a voltage between the measurement electrode and the reference electrode during storage of the probe.

5. A probe according to claim 1, in which the fluid medium is a molten metal, such as a metal comprising Al, Cu or Zn, or a molten glass.

6. A probe according to claim 1, in which the gas is hydrogen or oxygen.

7. A probe according to claim 1, in which the measurement chamber is hermetically sealed except at the porous wall portion and the purge-gas feed.

8. A probe according to claim 1, in which the purge-gas feed is closable for measurement of the concentration of the gas in the measurement chamber.

9. A probe according to claim 1, in which the purge-gas feed is openable to supply a volume of the purge gas to the measurement chamber, preferably before a measurement of the concentration of the gas is made.

10. A probe according to claim 1, in which the purge-gas feed is openable to supply a volume of purge gas of at least 50 ml, and preferably at least 100 ml or 200 ml or 300 ml or 500 ml (as measured at atmospheric pressure).

11. A probe according to claim 1, in which the purge gas is supplied at 1.7 ml·s⁻¹, or more, and preferably at 3.4 ml·s⁻¹, 5.0 ml·s⁻¹ or 6.7 ml·s⁻¹ or more (as measured at atmospheric pressure), optionally for a duration of 20 s to 60 s, or 30 s to 40 s.

12. A probe according to claim 1, in which the rate of flow of the purge gas through the area of the porous wall portion is at least 0.04 ml·s⁻¹·mm⁻², 0.08 ml·s⁻¹·mm⁻², 0.13 ml·s·⁻¹·mm⁻², or 0.16 ml·s⁻¹·mm⁻², or 0.2 ml·s⁻¹·mm⁻² (as measured at atmospheric pressure).

13. A probe according to claim 1, in which the purge-gas feed is openable to supply a continuous flow of the purge gas to the measurement chamber, preferably at a flow rate or pressure sufficient to prevent gas from passing into the measurement chamber through the porous wall portion, for example during storage of the probe.

14. A probe according to claim 13, in which the continuous flow of the purge gas is less than 10%, or 20%, or 50% greater than a minimum flow rate or pressure required to prevent gas from passing into the measurement chamber through the porous wall portion.

15. A probe according to claim 1, in which the purge gas comprises N₂ or an inert gas or a calibration gas.

16. A probe according to claim 1, in which the measurement chamber is defined at an end of a probe sleeve.

17. A probe according to claim 16, in which the wall defining the measurement chamber comprises an end portion of the probe sleeve.

18. A probe according to claim 16, in which the probe sleeve comprises a sleeve portion and an end cap which is secured, and optionally removably secured, to the end of the sleeve portion, the end cap comprising some or all of a wall defining the measurement chamber.

19. A probe according to claim 1, in which the purge-gas feed is connected by a purge-gas feeder channel, defined within the probe, to the measurement chamber, and in which a volume of the feeder channel is preferably less than twice a volume of the measurement chamber, and preferably less than 1.5 times, or 1.0 times, or 0.5 times, the volume of the measurement chamber.

20. A probe according to claim 19, in which the feeder channel is defined within the probe sleeve.

21. A probe according to claim 16, in which an inner surface of a wall of the probe sleeve defines the feeder channel.

22. A probe according to claim 16, in which the sensor is supported by or within the probe sleeve, being positioned so as to measure the concentration of the gas in the measurement chamber.

23. A probe according to claim 22, further comprising a sensor support, in which the sensor is supported at an end of the sensor support and the sensor support extends within, or along, at least a part of a length of the probe sleeve.

24. A probe according to claim 23, in which the sensor support extends along or within at least a quarter of the length of the probe sleeve, or at least along or within a portion of the probe sleeve which is, in use, immersed in the liquid.

25. A probe according to claim 23, in which the sensor support and the probe sleeve are located or fixed relative to one another at a point distant from, or spaced from, the measurement chamber, for example being located or fixed to each other or to a terminal block or to a handle or other support.

26. A probe according to claim 23, in which the feeder channel is at least partially defined between an inner surface of the probe sleeve and an outer surface of the sensor support.

27. A probe according to claim 1, in which the sensor is an electrolytic sensor having a measurement electrode and a reference electrode on opposite surfaces of a solid electrolyte, the measurement electrode being positioned within, or accessible to gas within, the measurement chamber, and the reference electrode being exposed, in use, to a reference concentration of the gas.

28. A probe according to claim 27, in which the reference concentration of the gas is provided by a solid reference standard or by a gaseous supply comprising the gas.

29. A probe according to claim 1, in which the measurement chamber is at an end of the probe and an electrical connection from the reference electrode and/or the measurement electrode extends within the probe.

30. A probe according to claim 16, in which the probe sleeve comprises, or consists of, a ceramic material or a metallic material.

31. A probe according to claim 1, in which the sensor is supported at an end of a tubular sensor support and an electrical connection from the reference electrode and/or the measurement electrode extends within the sensor support.

32. A probe according to claim 34, in which the sensor support comprises, or consists of, a metallic material.

33. A probe according to claim 23, in which, in use, the probe sleeve contacts the fluid medium and shields the sensor support from contact with the fluid medium.

34. A probe according to claim 1, further comprising a calibration-gas feed openable to supply to the measurement chamber a calibration gas containing a predetermined concentration of the measurand gas.

35. A probe according to claim 34, in which the purge-gas feed and the calibration-gas feed are the same component, selectively couplable to a source of the purge gas or a source of the calibration gas.

36. A probe according to claim 1, further comprising a heater for heating the sensor.

37. A probe according to claim 36, in which the heater is an electrical heater couplable to a power-supply voltage.

38. A probe according to claim 37, in which the heater comprises a thermocouple.

39. A probe according to claim 37, in which the sensor is an electrolytic sensor comprising a measurement electrode and a reference electrode, and in which a voltage is couplable between the measurement electrode and the reference electrode.

40. A probe according to claim 37, in which the power-supply voltage is the same as the voltage couplable between the measurement electrode and the reference electrode.

41. A probe according to claim 3, in which the heater is an electrical heater couplable to a power-supply voltage, preferably at a portion of the probe spaced from the electrolytic sensor.

42. A probe according to claim 41, in which the electrolytic sensor comprises a measurement electrode and a reference electrode on opposite surfaces of a solid electrolyte, and in which the power supply voltage is additionally couplable between the measurement electrode and the reference electrode.

43. A probe according to any of claims 3, in which the heater can raise the temperature of the electrolytic sensor to more than 50 C, 100 C or 150 C above ambient temperature.

44. A probe according to claim 2, in which the calibration gas comprises a predetermined concentration of the gas for measurement.

45. A method for operating a probe as defined in claim 2, comprising the step of supplying the calibration gas to the measurement chamber and using the sensor to measure a gas concentration in the measurement chamber.

46. A method for operating a probe as defined in claim 1, comprising the steps of;
supplying the volume of the purge gas through the purge-gas feed and the measurement chamber, so that the purge gas flows outwardly from the measurement chamber through the porous wall portion;
closing the supply of the purge gas or the purge-gas feed so as to seal the measurement chamber;
allowing a sampling time to pass, for the gas in solution in the fluid medium to pass through the porous wall portion into the measurement chamber; and
using the sensor to measure a concentration or partial pressure of the gas in the measurement chamber.

47. A method for measuring a concentration of a measurand gas in solution in a fluid medium, comprising the steps of;
supplying a purge gas to the measurement chamber such that a portion of the purge gas is forced out of the measurement chamber through the porous wall portion and such that the measurement chamber is filled with the purge gas;
closing a supply of the purge gas and allowing a sampling time to pass, during which time the measurand gas can pass through the porous wall portion into the measurement chamber; and
measuring a concentration, or partial pressure, of the measurand gas in the measurement chamber.

48. A method according to claim 46, in which the volume of purge gas is at least 50 ml, and preferably at least 100 ml or 200 ml or 300 ml or 500 ml (as measured at atmospheric pressure).

49. A method according to claim 46, in which the purge gas is supplied at 1.7 ml·s⁻¹ or more, and preferably at 3.4 ml·s⁻¹, 5.0 ml·s⁻¹, 6.7 ml·s⁻¹ or 8.3 ml·s⁻¹ or more (as measured at atmospheric pressure).

50. A method according to claim 46 in which the rate of flow of the purge gas through the area of the porous wall portion is at least 0.04 ml·s⁻¹·mm⁻², 0.08 ml·s⁻¹·mm⁻², 0.13 ml·s⁻¹·mm⁻², 0.16 ml·s⁻¹·mm⁻² or 0.2 ml·s⁻¹·mm² (as measured at atmospheric pressure).

51. A method according to claim 46, in which the sampling time is long enough for the gas to equilibrate in the measurement chamber.

52. A method for operating a probe as defined in claim 1, comprising the step of;
supplying a flow of the purge gas or other storage gas through the purge-gas feed during storage of the probe, at a flow rate or pressure sufficient to reduce diffusion of gas, such as atmospheric oxygen or water vapour, into the measurement chamber through the porous wall portion.

53. A method according to claim 52, in which the flow of the purge gas or other storage gas is no more than 10% or 20% or 50% greater than a minimum flow rate or pressure required substantially to prevent gas from passing into the measurement chamber through the porous wall portion.

54. A method for operating a probe as defined in claim 1, comprising heating at least the sensor during storage of the probe, or when the probe is not being used for measurement.

55. A method according to claim 54, comprising the step of heating at least the sensor before a gas measurement is made, preferably for between 1 minute and 10 minutes.

56. A method according to claim 54, comprising the step of heating the sensor to more than 50 C, 100 C, 150 C or 200 C, or into a temperature range of 50 C to 200 C or 80 C to 150 C.

57. A method for storing a probe as defined in claim 4, comprising applying a voltage between the measurement electrode and the reference electrode, preferably of between 3 V and 20 V, or 6 V and 15 V, or 8 V and 13 V.

58. A prove sleeve as defined in claim 1.

59. (canceled)

60. (canceled)

61. (canceled)
